(19) United States
(12) Patent Application Publication
(10) Pub. No.: US 2004/0023319 A1
(43) Pub. Date: Feb. 5, 2004

(54) Rapid enumeration of viable spores by flow cytometry

(76) Inventors: William L. Godfrey, Danville, CA (US); Rana A. Alsharif, San Jose, CA (US)

Correspondence Address: BECTON, DICKINSON AND COMPANY, 1 BECTON DRIVE, FRANKLIN LAKES, NJ, 07417-1880, US

(21) Appl. No.: 10/390,295

(22) Filed: Mar. 17, 2003

Related U.S. Application Data
(60) Provisional application No. 60/367,651, filed on Mar. 26, 2002.

(51) Int. Cl. C12Q 1/04

(52) U.S. Cl. 435/34

(57) ABSTRACT

Methods for determining the number of viable spores in a sample are provided. In practicing the subject methods, aliquots of a sample are stained with a combination of a permeant and an impermeant dye both before and after a germination step, and then analyzed by flow cytometry. By comparing the results obtained before and after germination, the number of viable spores in the sample is readily determined. Also provided are kits and systems for use in practicing the subject methods. The subject methods and compositions find use in a variety of different applications where it is desirable to determine the number of viable spores in a sample.

This application claims priority under 35 U.S.C. § 119(e) of provisional application Serial No. 60/367,651, filed Mar. 26, 2002, which is incorporated herein by reference.

### FIELD OF THE INVENTION

The field of this invention is flow cytometry.

### BACKGROUND OF THE INVENTION

Spore quantitation is used in a wide variety of microbiological applications, including use of biological indicators and identification of environmental contaminants. Viable spores are of particular interest as these give rise to vegetative cells. Currently, viable spore counts are done by limited dilution culture on plates and counting of the resulting colonies. The process generally requires 24 or more hours for culture and considerable operator time to acquire statistically significant counts.

Relevant Literature

U.S. patents of interest include: U.S. Pat. Nos. 5,701,012; 5,795,730; 5,876,960; 5,895,922; and 6,228,574. U.S. patents describing flow cytometry include: U.S. Pat. Nos. 4,704,891; 4,727,029; 4,745,285; 4,867,908; 5,342,790; 5,620,842; 5,627,037; 5,701,012; 5,895,922; 5,981,180; 6,159,748 and 6,287,791. Also of interest is published United States patent application no. 20010008217. Other references of interest include: Bailey et al., Science (1977) 198:1175-1176; Phillips & Martin, Cytometry (1983) 4:123-131; Phillips & Martin, Cytometry (1985) 6:124-129; Stopa, Cytometry (2000) 41:237-244.

### SUMMARY OF THE INVENTION

The present invention provides methods and reagents for detecting viable spores in a sample. The methods provide both qualitative detection methods, i.e., screening for the presence of viable spores, and quantitative detection methods, i.e., determining the number or concentration of the viable spores.

In the present methods, a first aliquot of a sample is stained with a combination of a permeant dye and an impermeant dye to label live and dead organisms. A second aliquot of the sample is first subjected to germination conditions, and then is stained as with the first aliquot. The two aliquots of the sample are analyzed by flow cytometry. Spores that have germinated into vegetative cells when subjected to germination conditions can be distinguished from ungerminated spores by their staining pattern. By comparing the results obtained from the flow cytometric analysis, the presence, number, or concentration of viable spores in the sample is readily determined.

Thus, one aspect of the invention relates to methods of detecting viable spores in a sample, said methods comprising:

a) contacting an aliquot of the sample with a permeant dye and an impermeant dye to produce a stained ungerminated sample;

b) treating an second aliquot of the sample to germination conditions,

c) contacting the second aliquot with a permeant dye and an impermeant dye to produce a stained germinated sample;

d) flow cytometrically analyzing the stained ungerminated sample to obtain a ungerminated data set;

e) flow cytometrically analyzing the stained germinated sample to obtain a germinated data set; and

f) comparing the ungerminated and germinated data sets to detect viable spores in said sample.

In some embodiments of the invention, the methods provide a qualitative detection of viable spores in the sample, which is useful for screening a sample for the presence of viable spores. In other embodiments of the invention, the methods provide a quantitative detection of viable spores in the sample, which is useful of determining either the number or concentration of viable spores.

In another aspect, the present invention provides methods for determining the effectiveness of a sterilization process, wherein viable spores are detected, preferably quantitatively, following sterilization, using the methods of the invention. The effectiveness of the sterilization process is measured by the decrease in viable spores detected following sterilization. Preferably, a control sample, typically consisting of an untreated aliquot of the original sample, is analyzed to facilitate a reliable determination of the decrease in viable spores.

In another aspect, the present invention provides kits for use in practicing the subject methods, comprising a permeant dye, an impermeant dye; and, optionally, other reagents useful for carrying out the present methods, such as a substrate comprising instructions for practicing the methods, a calibration composition, or a surfactant.

The subject methods and compositions find use in a variety of different applications where it is desirable to determine the number of viable spores in a sample. The methods enable the rapid and accurate determination of the viable spore counts in a sample and, furthermore, can provide results within several hours with large numbers of spores.

### BRIEF DESCRIPTION OF THE FIGURES

FIG. 1A provides an FSC-H vs. SSC-H dot plot showing regions selected to encompass Bacillus subtilis cells (R1) and control microbeads (R2), as described in Example 2.

FIG. 1B provides an FL1-H vs. FL3-H dot plot of Bacillus subtilis stained with a permeant and an impermeant dye, with the sample containing about equal amounts of live and killed bacteria, as described in Example 2.

FIGS. 2A and 2B provide FL1-H vs. FL3-H dot plots of Bacillus subtilis stained with a permeant and an impermeant dye, as described in Example 3. FIG. 2A provides the results obtained from a sample containing spores and killed bacteria. FIG. 2B provides the results obtained from a sample containing spores, killed bacteria, and live, vegetative cells.

FIGS. 3A, 3B, 3C, 3D, and 3E provide FL1-H vs. FL3-H dot plots of Bacillus subtilis (spores and killed cells) stained with a permeant and an impermeant dye after treatment under germination conditions for 0, 1, 2, 3, and 4 hours, respectively, as described in Example 4.

### DESCRIPTION OF THE SPECIFIC EMBODIMENTS

The following definitions are provided to facilitate understanding of the invention, and are not intended to limit the invention to the particular embodiments described below. Unless defined otherwise, all technical and scientific terms used herein have the same meaning as commonly understood to one of ordinary skill in the art to which this invention belongs.

In this specification and the appended claims, the singular forms “a,” “an” and “the” include plural reference unless the context clearly dictates otherwise.

As used herein, the term “ungerminated” refers to a sample that has not been subjected to germination conditions. The term refers to the lack of treatment of the sample, and is not meant to indicate the presence or absence of germinated cells.

As used herein, the term “germinated” refers to a sample that has been subjected to germination conditions. The term refers to the treatment of the sample, and is not meant to indicate the presence or absence of germinated cells.

As used herein, the term “permeant dye” refers to a light emitting compound that is capable of permeating cell membrane walls and binding to an intracellular molecule. Preferably, a permeant dye is a fluorescent nucleic acid binding compound.

As used herein, the term “impermeant dye” refers to a light emitting compound that cannot traverse living cellular membranes, but can traverse the disrupted membranes of injured or dead cells and bind to an intracellular molecule. Preferably, an impermeant dye is a fluorescent nucleic acid binding compound.

Where an exemplified experiment is described as carried out “essentially as” a given protocol, it is understood that the actual protocol used may contain one or more differences from the protocol described, but that the differences are believed to be minor and not likely to affect the qualitative results of the experiment or alter the conclusion drawn.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein are incorporated herein by reference for the purpose of describing and disclosing components that are described in the publications which might be used in connection with the presently described invention.

The preferred methods and compositions are described below in greater detail. However, it will be clear that any methods and compositions similar or equivalent to those described herein can be used in the practice or testing of the invention.

Methods

In practicing the subject methods, the following steps are typically practiced: 1) sample preparation; 2) staining of ungerminated sample; 3) flow cytometric analysis of ungerminated sample; 4) germination; 5) staining of germinated sample; 6) flow cytometric analysis of germinated sample; and 7) data analysis/processing. It will be understood that the order of the steps may be varied for convenience. For example, the ungerminated sample may be held until after the germination step, and then stained and analyzed at the same time as the germinated sample. Each of these general steps is described in greater detail, below.

Sample Preparation

The present methods may be used essentially with any sample for which a qualitative or quantitative determination of viable spores therein is desired, and that is amenable to analysis by flow cytometry. Some samples, such as fluid suspensions of cells, may be used directly or with appropriate dilution or concentration. Other samples, such a sample from a non-fluid source, e.g., a solid or aerosol, may require processing in some manner to provide a suitable sample for analysis by flow cytometry. Sample preparation methods suitable for use in flow cytometry are well known in the art.

Samples of interest include, but are not limited to: industrial fluids, e.g., spore suspensions used for sterility challenge or process validation; pharmaceutical samples; biological fluids, e.g., blood, tears, or saliva; biological solids that are treated to provide fluid samples, e.g., tissues or organs that are subjected to one or more processing steps, e.g., homogenization, to provide a fluid sample; environmental fluids, e.g. surface water or ground water; environmental solids, e.g., soils, etc, that are combined with a fluid to provide a fluid sample; and aerosols that are combined with a carrier fluid to produce a fluid sample (see, e.g., U.S. Pat. Nos. 5,701,012 and 5,895,922, which discuss preparation of aerosol samples for use in flow cytometers, the disclosures of which are herein incorporated by reference).

For use with samples such as pharmaceutical, food, or environmental samples, at least 100 organisms per ml are required to be detected using flow cytometry. If necessary, samples can be brought into this range by an initial concentration step.

For many organisms, the initial sample may be incubated at 60° C. to 80° C. for 10 minutes to 20 minutes, or by a similar process, to kill vegetative cells present, without killing viable spores.

Staining of Ungerminated Sample

A portion of the ungerminated sample is labeled or stained by contacting the sample with both a permeant dye and an impermeant dye.

Permeant dyes are known to those of skill in the art. Preferred permeant dyes are permeant fluorescent nucleic acid binding compounds that exhibit an increase in fluorescence upon binding to nucleic acid, such as, for example, thiazole orange and analogs thereof (such as those described in U.S. Pat. Nos.: 4,883,867; 4,957,870; 5,656,449; each incorporated herein by reference); SYTO® dyes, e.g., SYTO® 16, SYTO® 59 (described in U.S. Pat. Nos. 5,436,134 and 5,534,416, both incorporated herein by reference), and the like. The SYTO® dyes and additional permeant dyes are available from Molecular Probes (Eugene, Oreg.). The concentration of permeant dye that is contacted with the sample may vary depending, e.g., on the particular nature of the dye, but in many embodiments ranges from about 50 nM to about 2 μM, usually from about 100 nM to about 500 nM for a sample volume of 200 μl.

Impermeant dyes are well known in the art. Preferred impermeant dyes are impermeant fluorescent nucleic acid binding compounds that exhibit an increase in fluorescence upon binding to nucleic acid, such as, for example, propidium iodide, ethidium bromide, and the like. Additional impermeant dyes are available from Molecular Probes (Eugene, Oreg.). The concentration of impermeant dye that is contacted with the sample may vary depending, e.g., on the particular nature of the dye, but in many embodiments ranges from about 1 μM to about 1 mM, usually from about 10 μM to about 100 μM for a sample volume of 200 μl.

The sample may be contacted with one or more surfactants that act to enhance the ability of the permeant and impermeant dyes to reach their nucleic acid binding targets. Examples of suitable surfactants include, but are not limited to: 1) polyoxyethylene-sorbitan-fatty acid esters, e.g. mono- and tri-lauryl, palmityl, stearyl and oleyl esters, e.g., of the type known and commercially available under the trade name TWEEN™, including the products:

TWEEN™ 20 [polyoxyethylene(20)sorbitanmonolaurate],

TWEEN™ 40 [polyoxyethylene(20)sorbitanmonopalmitate],

TWEEN™ 60 [polyoxyethylene(20)sorbitanmonostearate],

TWEEN™ 65 [polyoxyethylene(20)sorbitantristearate],

TWEEN™ 85 [polyoxyethylene(20)sorbitantrioleate],

TWEEN™ 21 [polyoxyethylene(4)sorbitanmonolaurate],

TWEEN™ 81 [polyoxyethylene(5)sorbitanmonooleate], etc.; and 2) poloxyethylene-polyoxypropylene co-polymers and block co-polymers, e.g., of the type known and commercially available under the trade name PLURONIC® (BASF, Mt. Olive, N.J.), such as PLURONIC® F68; and the like.

In addition, a chelator may be contacted with the sample in order to open up the lipopolysaccharide layer on cells in the sample, e.g., on gram-negative bacteria. Chelators of interest include, but are not limited to, EDTA, EGTA, and the like.

In addition, the solution in contact with the sample should be buffered to a range of 5.5 to 8.5. Buffering salts of interest include, but are not limited to: sodium phosphate; sodium citrate; and the like. Buffers may comprose combinations of salts to achieve the desired pH.

In addition, the solution in contact with the sample may contain salts to make it isotonic. Such salts include, but are not limited to: sodium chloride; and the like.

The sample may be contacted with the permeant and impermeant dyes, as well as the additional optional reagents described above, using any convenient protocol, so long as the sample and dyes are contacted under conditions such that the permeant dye can enter live and dead cells and the impermeant dye can enter dead cells. Typically, contact of the sample with the above agents is performed under incubation conditions that provide for staining of the spores and cells in the sample. Typically, the labeling reagents and samples are contacted and combined at a temperature ranging from about 2° C. to about 50° C., usually from about 18° C. to about 40° C. Contact typically is performed with mixing or agitation, e.g., with vortexing, etc., to provide for sufficient combination of the labeling reagents and the sample. The sample typically then is maintained or incubated for a period of time prior to flow cytometric analysis, where the sample is generally incubated at a temperature ranging from about 2° C. to about 50° C., usually from about 18° C. to about 40° C. for a period of time ranging from about 2 minutes to about 60 minutes, usually from about 10 minutes to about 30 minutes. Optionally, the sample may be washed following incubation to remove excess dye.

Following the above incubation step, the sample preferably is assayed immediately. Storage of the prepared sample may affect the results of the assay.

Flow Cytometric Analysis of Ungerminated Sample

Once the sample has been prepared as described above by staining with a permeant and impermeant dye, the sample is flow cytometrically analyzed to detect the presence of spores in the sample, either qualitatively or quantitatively.

For quantitative determinations, a calibration standard may be added to the sample in order to obtain the absolute count (concentration) of the analyzed particles. A preferred calibration standard consists of a fluorescent microparticle that typically ranges in size between 2 μm and 10 μm in diameter, and is made of a material that avoids clumping or aggregation. Fluorescence can be achieved by selecting microparticles comprised of an autofluorescent material or labeled (tagged) with fluorescent dye. The microparticles are selected such that the fluorescence is detectable over background noise and is distinguishable from the fluorescence of the impermeant and permeant dyes. In general, a one log difference (i.e., 10-fold) in the measured fluorescence intensity is sufficient to distinguish objects in one of the fluorescence channels of a flow cytometer.

Microparticles having these properties may be selected from the group consisting of fixed chicken red blood cells, dye-incorporated beads (e.g., coumarin beads, fluorescein beads, rhodamine beads), liposomes containing a fluorescent dye, fixed fluorescent cells, fluorescent cell nuclei, and fluorescent microorganisms. For greatest precision, the concentration of the microparticle should be greater than or, preferably, equal to the number of cells to be counted. Although a 1:1 ratio of microparticles to cells is preferred, usable results can be obtained with ratios between about 10:1 and about 1:10. A variety of such calibration beads and protocols for their use in obtaining absolute cell counts via flow cytometry are known and commercially available. Representative calibration products include, but are not limited to, TruCOUNT™ Tubes, TruCOUNT™ controls (BD Biosciences, San Jose, Calif.), and the like.

Absolute counts also may be obtained using alternative protocols, such as spiking in a counted liquid bead suspension, driving the sample through the instrument by syringe, or other metered positive-displacement means.

The amount of sample that is assayed is generally small, typically ranging from about 100 μL to about 2 ml, usually from about 200 μl to about 1 ml.

Flow cytometry is a well-known methodology using multi-parameter data for identifying and distinguishing between different particle types (spores, cells, or beads) in a sample. For flow cytometric analysis of the sample prepared as described above, the sample is first introduced into the flow path of the flow cytometer, and the spores and cells present in the flow path are passed substantially one at a time through one or more sensing regions. The individual particles are exposed separately to a source of light at essentially a single wavelength, and measurements of typically at least two light scatter parameters and one or more fluorescent emissions are recorded separately for each particle. The data recorded for each particle are analyzed in real time or stored in a data storage and analysis means, such as a computer. U.S. Pat. No. 4,284,412, incorporated herein by reference, describes the configuration and use of a typical flow cytometer equipped with a single light source, while U.S. Pat. No. 4,727,020, incorporated herein by reference, describes the configuration and use of a flow cytometer equipped with two light sources.

The source of light generally comprises an illumination means that emits light of essentially a single wavelength, such as a laser (e.g., argon or He/Ne), light-emitting diode, or a mercury arc lamp with appropriate filters. Light at 488 nm is a generally used excitation wavelength in a flow cytometer having a single light source. For flow cytometers that excite with light at two distinct wavelengths, additional wavelengths of emission light that are commonly employed include, but are not limited to: 535 nm; 635 nm; and the like.

In series with a sensing region, multiple light collection means, such as photomultiplier tubes (or “PMTs”), are used to record light that scatters forward at a low angle for each particle (generally referred to as forward light scatter), light that is reflected orthogonal to the direction of the flow of the particles through the sensing region (generally referred to as orthogonal or side light scatter) and fluorescent light emitted from the particle, if it is labeled with fluorescent marker(s), as the particle passes through the sensing region and is illuminated by the light source. Detection by a PMT of light only in a desired range of wavelengths is achieved by the use of appropriate filters in the light path. Each of forward light scatter (or FSC), orthogonal light scatter (SSC), and fluorescence emissions (FL1, FL2, FL3, etc., referring to different wavelength ranges) comprise a separate parameter for each spore/cell/bead (or each “event”). Typically, the magnitude of each parameter, generally measured as the height of the voltage pulse (indicated by a suffixed “H”, e.g., FL1-H), is recorded for each particle.

Flow cytometers further include data acquisition, analysis and recording means, such as a computer, wherein multiple data channels record data from each PMT for the light scatter and fluorescence emitted by each particle as it passes through the sensing region. The purpose of the analysis system is to classify and count events, wherein each event presents itself as a set of digitized parameter values.

In analyzing the sample as described above, the flow cytometer typically is set to record data only for events that exceed a preset threshold value in a selected parameter. This “trigger” on a preset threshold allows data acquisition selectively for true events, thereby reducing background and noise. Typically, triggering is used to detect passage of a cell or other particle of interest based on forward-scattered or side-scattered light, with the threshold set to identify particles of the expected size and/or granularity. Detection of a particle triggers acquisition of light scatter and fluorescence data for the particle by the flow cytometer.

Selective analysis of a subpopulation of particles may be achieved by “gating” based on a subset of the measured parameters. The data collected for the entire population of particles are plotted so as to obtain the best possible separation of subpopulations, e.g., spores compared to other entities present in the sample, such as vegetative and dead bacterial cells. A gate then is defined on the plot as a region encompassing the desired subpopulation. Further analysis is carried out only for those events within the gate; events which are not within the gate are ignored. Gating to allow selective analysis of spore, vegetative, and bead subpopulations typically is carried out by plotting forward light scatter (FSC) vs. side (i.e., orthogonal) light scatter (SSC) and SSC vs. FL2 on two-dimensional dot plots.

Flow cytometric analysis of the ungerminated sample, as described above, yields qualitative and quantitative information about the presence of the spores in the ungerminated sample being assayed, or, equivalently, in the sample prior to germination. The data obtained from the flow cytometric analysis of the ungerminated sample are referred to herein as the “ungerminated data set.”

Germination

An aliquot of the sample is subjected to germination conditions sufficient to cause germination of viable spores present in the sample. Any conditions that allow rapid spore germination may be employed. Typically, germination is carried out by contacting the sample with a germination medium followed by incubation under suitable conditions for a period of time that provides for spore germination. A representative germination medium is brain-heart infusion medium, a well known and commercially available microbial growth medium that contains infusions of both calf brain and beef heart and is used to culture a range of bacteria and fungi. The contacted sample is typically incubated with the germination medium for a period of time ranging from about 2 hours to about 24 hours, usually from about 3 hours to about 4 hours, at a temperature ranging from about 35° C. to about 40° C.

Many other germination protocols are known to those of skill in the art and may be employed, where representative protocols include those described in U.S. Pat. Nos. 5,795,730 and 6,228,574; the disclosures of which are herein incorporated by reference.

Staining of Germinated Sample

Following the above germination step, the germinated sample is stained by contact with a permeant dye and an impermeant dye, essentially as described for the ungerminated sample.

Flow Cytometric Analysis of Germinated Sample

The stained germinated sample is analyzed by flow cytometry, essentially as described for the ungerminated sample, to obtain data, both qualitative and quantitative, regarding the number of remaining spores, as well as the number of live vegetative cells. The data obtained from the flow cytometric analysis of the germinated sample are referred to herein as the “germinated data set.”

Data Processing/Analysis

In this final step of the subject methods, the obtained ungerminated data set and germinated data set are compared to detect, in the broadest sense, viable spores in the sample. Comparison typically includes comparing the ungerminated and germinated data sets to identify a decrease in the detected spore population and an increase in the vegetative cell population when the sample includes viable spores.

In certain embodiments, viable spore concentrations are obtained in this step of the subject methods. To obtain viable spore concentrations, the concentration of spores detected in the germinated data set is subtracted from the concentration of spores detected in the ungerminated data set in order to obtain a concentration of the viable spores in the assayed sample. Using these values, viable spore counts can also be expressed as a percent of total spores.

Using the above methods, one can obtain rapid detection of viable spores in a sample with large numbers of analyzed spores. Typically, results are obtained in a period of time (encompassing germination, staining, and analyis) that is less than about 5 hours, often less than about 4 hours. Within the analysis time period, generally less than one minute, large numbers of spores can be analyzed, where by large numbers is meant up to about 5000 or more spores, where in many embodiments, the subject methods are limited to the number of colonies that can be counted and analyze from about 100 to about 1000 spores.

Utility

The subject methods find use in a variety of applications where viable spore counts in a sample are desired, where such applications include microbiological applications, sample screening applications, e.g., screening samples for environmental hazards, including biohazardous spores; sterility screening assays in which a particular sterilization protocol is assayed for its ability to sterilize a given spore containing sample (see e.g., U.S. Pat. No. 5,795,730, incorporated herein by reference) and the like.

The subject methods can be employed to analyze practically any type of spore-forming species, including both prokaryotic and eukaryotic spore-forming species. Representative spore-forming species that may be analyzed according to the subject invention include bacteria, e.g., bacilli and the like, and fungi, e.g., yeast and the like.

It is evident from the results provided in the examples, below, and discussion herein, that the subject invention provides for a number of distinct advantages with respect to the analysis of viable spore counts in samples. Advantages include the rapidity of the methods; the possibility of analyzing large numbers of spores in short periods of time, providing improved count statistics; the ability to decrease variability by converting manual evaluation to instrument-based protocols; and, potentially, the ability to identify “viable-but-not-culturable” spores, a population that has not been identifiable to date. As such, the subject invention represents a significant contribution to the art.

Kits

In yet another aspect, the present invention provides kits for practicing the subject methods. The subject kits at least include at least one permeant dye and at least one non-permeant dye, as described above. In addition, the kits may include calibration beads, as described above. In addition, the kit may include one or more additional compositions that are employed, including but not limited to: buffers, diluents, media, etc., which may be required to produce a fluid sample from an initial non-fluid, e.g., aerosol or solid sample, or to otherwise prepare an initial fluid sample for analysis, e.g., enrich or dilute a sample with respect to the analytes of interest; surfactant(s); chelator(s); and the like.

The above components may be present in separate containers or one or more components may be combined into a single container, e.g., a glass or plastic vial. Of particular interest in certain embodiments are kits that include a single container that includes at least the calibration beads, when present, and serves as a sample preparation container, e.g., into which sample may be added as well as dyes and other reagents. In certain embodiments, the dye and other reagents may also be present in the container such that a single container contains all necessary reagents and one need just add sample to the container in order to prepare and label the sample for flow cytometric analysis.

In addition to the sample preparation reagents described above, the subject kits may also include a germination medium/agent.

The subject kits also may include a computer-readable medium, e.g., diskette, CD, etc., containing programming for use in automating the analysis of samples and evaluation of the results.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Systems

Also provided are systems for use in practicing the subject methods. The subject systems include the various reagent components required to perform the assay, e.g., permeant and impermeant dyes, as well as a flow cytometric detector. The subject systems also may include a computer readable medium, e.g., diskette, CD, etc., containing programming for use in automating the analysis of samples and evaluation of the results. Representative flow cytometric devices include, but are not limited to those devices described in U.S. Pat. Nos. 4,704,891; 4,727,029; 4,745,285; 4,867,908; 5,342,790; 5,620,842; 5,627,037; 5,701,012; 5,895,922; and 6,287,791; the disclosures of which are herein incorporated by reference.

The examples of the present invention presented below are provided only for illustrative purposes and not to limit the scope of the invention. Numerous embodiments of the invention within the scope of the claims that follow the examples will be apparent to those of ordinary skill in the art from reading the foregoing text and following examples.

### EXAMPLE 1

### Preferred Protocols

Staining

Staining is carried out by incubating the sample in a staining buffer containing both a permeant dye and an impermeant dye. Sample should be diluted or concentrated, as appropriate, such that the concentration of organism to be detected is in a range suitable for analysis by flow cytometry. In general, at least 100 organisms/ml are required for detection by flow cytometry. For the analysis of cultured bacteria, the sample generally is diluted in staining buffer to an approximate concentration of 1×10⁵ to 1×10⁶ bacteria/ml.

A preferred permeant dye is thiazole orange, commercially available in a 17 μM solution (BD Biosciences, San Jose, Calif., Catalog No. 349483). Thiazole orange is hydrophobic; stock solutions should be maintained in DMSO or alcohol.

A preferred impermeant dye is propidium iodide solution, commercially available from a number of sources. Preferred is a 1.9 mM solution available from, for example, BD Biosciences (San Jose, Calif., Catalog No. 349483).

A preferred staining buffer consists of phosphate-buffered saline (PBS), 1 mM EDTA, 0.2% PLURONIC® F-68 (BASF Corporation, Mount Olive, N.J., Catalog No. 51554728), pH 7.4. TWEEN™ 20 at 0.01% can be substituted for PLURONIC® F-68. With some spores, the use of the PBS/TWEEN™ 20 buffer may result in slightly faster staining. The staining buffer preferably is passed through a 0.22-μm filter prior to use.

The bacterial suspension or sample is vortexed and then diluted at least 1:1, preferably 1:10, in staining buffer. A 200 μl aliquot of diluted bacterial suspension is added to a TruCOUNT™ Tube (BD Biosciences, San Jose, Calif., Catalog No. 340567), which contains a predetermined number of fluorescent microbeads for use as a quantitation standard. To the sample tube are added 5.0 μl of each dye solution. The final staining dye concentrations should be about 420 nM thiazole orange and 48 μM propidium iodide. The dye concentrations may be doubled with little affect on the staining. The mixture is vortexed and incubated to allow staining.

Optimal incubation time and temperature using these dyes depends on the organism. Essentially complete staining of bacteria such as Bacillus species is achieved with an incubation of 15 minutes at room temperature; staining for longer periods does not enhance fluorescence further. Staining carried out at 37° C. is more rapid and may be necessary for some spores.

Flow Cytometry

Flow cytometry is carried out using a BD FACS™ brand flow cytometer equipped with 488-nm laser excitation (BD Biosciences, San Jose, Calif.). Data are acquired from the prepared samples using an SSC threshold, using BD CellQuest™ or BD LYSYS™ II software in Acquisition-to-Analysis mode.

Using a FACSCalibur™ flow cytometer, the fluorescence channels are measured in the following ranges of wavelengths:

| Channel | Wavelength Range |
|---|---|
| FSC | 488 ± 10 nm |
| SSC | 488 ± 10 nm |
| FL1 | 500-560 nm |
| FL2 | 560-626 nm |
| FL3 | ≧670 nm |

Flow Cytometer Setup

CaliBRITE™ 3 beads (BD Biosciences, San Jose, Calif.) and software such as BD FACSComp™ or BD AutoCOMP™ are used for setting the photomultiplier tube (PMT) voltages and the fluorescent compensations, and for checking instrument sensitivity prior to use. The initial instrument settings are as follows:

| Threshold | SSC |
|---|---|
| FSC | E01, logarithmic amplification |
| SSC | 375 V, logarithmic amplification |
| FL1 | 600 V, logarithmic amplification |
| FL3 | 800 V, logarithmic amplification |
| Compensation | none used |

Setting FSC and SSC on logarithmic amplification assures that a wide range of bacterial sizes can appear on-scale and helps present recognizable populations for gating. Actual settings can vary depending on the application and should be optimized as described below.

The threshold on side scatter (SSC) is set, and the PMT voltages and threshold levels are adjusted, using an unstained sample of diluted bacteria so that the bacterial population is positioned entirely on scale on an FSC vs. SSC plot. Individual FSC and SSC histograms should be checked to be sure that the bell-shaped populations are not cut off on the display. If the entire population is not present, the PMT values are adjusted to position the peak on the histograms and decrease the threshold until the entire population is visible. As the voltage is further increased, the background noise should become evident on the lower end of the histogram. The PMT voltage and threshold should be balanced to allow the entire peak to be observed with at least a portion of the valley between the bacteria and the noise. Actual peak shapes and resolution from noise will vary with bacterial morphology and sample matrix.

The FL1 and FL3 PMT voltages are set to place the unstained population in the lower left quadrant of an FL1 vs. FL3 plot.

Gating

On a FSC vs. SSC dot plot, a region R1 is set liberally around the bacterial population, including spores and vegetative cells. If a quantitation bead is included in the sample, such as by the use of TruCOUNT™ Tubes, a region R2 is set around the beads.

On a FL2 vs. SSC dot plot, a region R3 is set around the stained bacteria.

The stained populations are visualized on an FL1 vs. FL3 dot plot, gated on (R1 AND R3).

Note that the permeant dye, thiazole orange, fluoresces primarily in FL1 and FL2, whereas the impermeant dye, propidium iodide, fluoresces primarily in FL3. Thus, region R3 is defined to include all bacteria stained by the permeant dye, i.e., all bacteria, whether live, dead, or a spore. Furthermore, the best discrimination of live cells, dead cells, and spores based on the relative staining by the permeant and impermeant dyes is obtained on an FL1 vs. FL3 plot.

Quantitation

The concentration of each cell type is determined from the data using the following equation:

Concentration=E/C×B/V×D,

where

E is the number of events in region containing cells of interest,

C is the number of events in the bead population (events in R2),

B is the total number of control beads used in the test*,

V is the volume of the sample analyzed, and

D is a sample dilution factor. *The number of control beads in a TruCOUNT™ Tube, which may vary from lot to lot, is indicated on the product label.

### EXAMPLE 2

### Discrimination of Live and Dead Cells

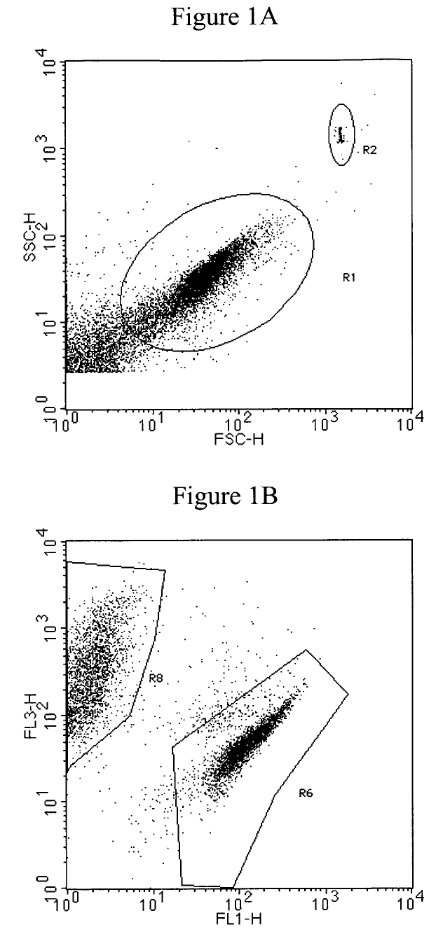

A suspension of live (vegetative) Bacillus subtilis in Trypticase™ soy broth (BD Diagnostic Systems, Sparks, M.D., Catalog No. 221092) was combined with an approximately equal amount of a suspension of dead Bacillus subtilis cells, and the resulting mixture was stained and analyzed by flow cytometry essentially as described in Example 1. Killed bacteria were prepared by mixing 0.5 mL of culture before dilution with 0.5 ml of SPOR-KLENZ™ (Steris Corporation, St. Louis, Mo., Catalog No. 6525-01) disinfectant for 5 minutes. No spores were present in the sample in this experiment. The results are provided in FIG. 1.

FIG. 1A shows an FSC-H vs. SSC-H dot plot with regions encompassing bacterial cells (R1) and microbeads (R2) indicated. These regions are used in the gating, as described in Example 1. The events counted from region R2 also provide the number of events in the bead population, used in the quantitation equation.

FIG. 1B shows an FL1-H vs. FL3-H dot plot, gated as described in Example 1, with regions encompassing the live cells (R6) and dead cells (R8) indicated. Regions R6 and R8 are disjoint, which shows that the differential staining of live and dead cells enables the two populations to be clearly distinguished. A small population (about 3% of the cells) of injured cells can be seen in the plot between regions R6 and R8.

### EXAMPLE 3

### Discrimination of Spores, Vegetative, and Dead Cells

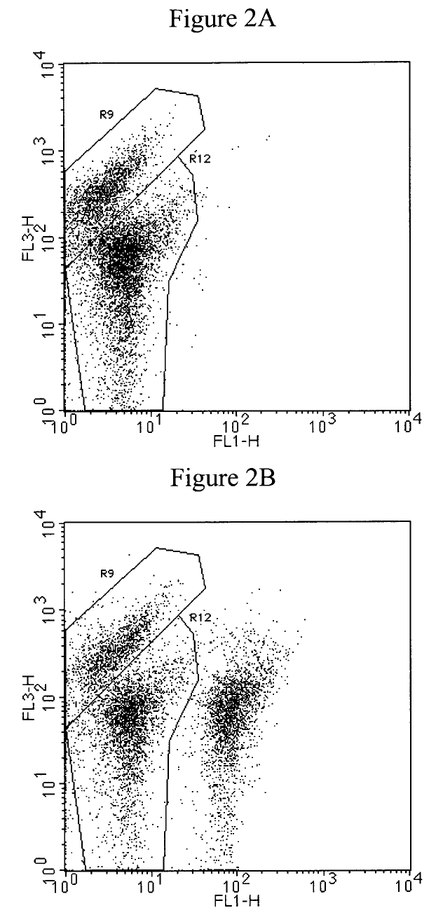

A suspension in water of about 10⁶ viable Bacillus subtilis spores/ml, as determined by plate count, was heated at 80° C. for 10 minutes to kill any residual vegetative bacteria. The sample was stained and analyzed essentially as described in Example 1. The results are shown in FIG. 2A.

FIG. 2A shows an FL1-H vs. FL3-H plot with regions encompassing dead cells (R9) and spores (R12) indicated. Regions R9 and R12 are disjoint, which shows that the differential staining of spores and dead cells enables the two populations to be clearly distinguished. In comparison with the results of Example 2, spores and dead cells are not as separated in the data space as are live cells and dead cells, although clear discrimination still is achieved.

In a second experiment, a suspension in water of about 106 viable Bacillus subtilis spores/ml, as determined by plate count, was heated at 80° C. for 10 minutes to kill any residual vegetative bacteria. To this sample was added an approximately equal quantity of live, vegetative cells. The sample was stained and analyzed essentially as described in Example 1. The results are shown in FIG. 2B.

FIG. 2B shows an FL1-H vs. FL3-H plot with regions encompassing dead cells (R9) and spores (R12) indicated. Regions R9 and R12 are adjacent, but disjoint, as in FIG. 2A. Live cells appear in a quite distinct region (not indicated in the plot) of the data space, well separated from both the dead cells (consistent with the results of Example 1) and the spores. The results show that the differential staining of vegetative cells, spores, and dead cells enables the three populations to be clearly distinguished.

### EXAMPLE 4

### Quantitative Detection of Viable Spores in a Sample

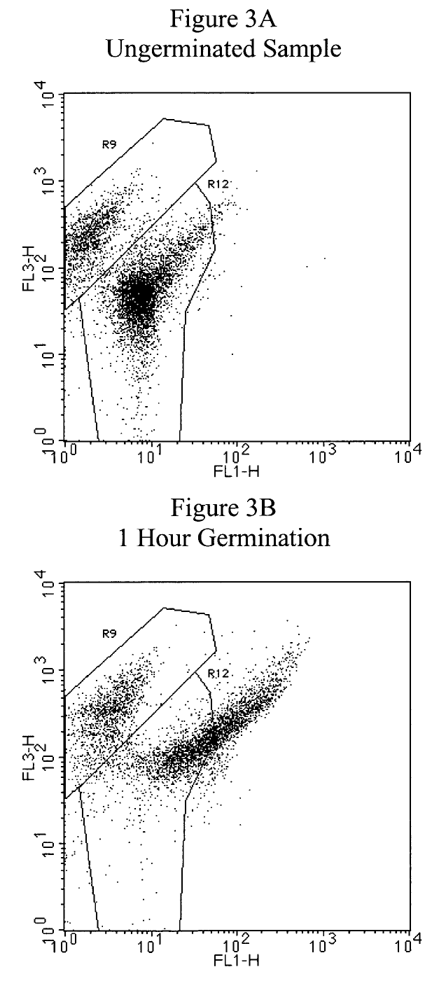

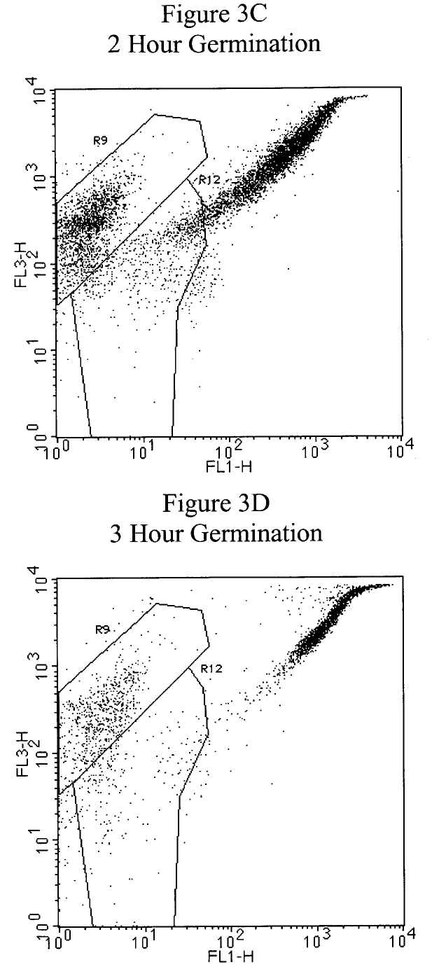

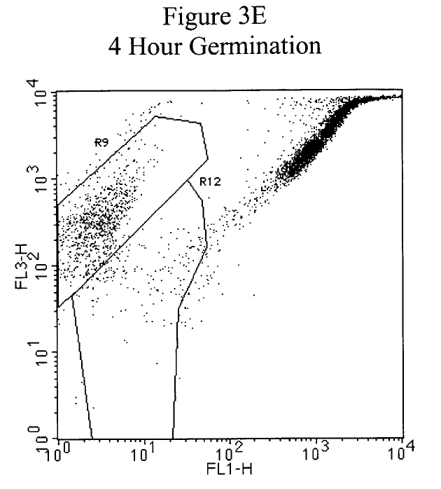

A suspension in water of about 1.3×10⁶ viable Bacillus subtilis spores/ml, as determined by plate count, were heated at 80° C. for 10 minutes to kill any residual vegetative bacteria. An aliquot of the untreated (ungerminated) suspension was stained and analyzed essentially as described in Example 1. The results from the ungerminated sample, shown as an FL1-H vs. FL3-H plot, are shown in FIG. 3A.

The remaining sample was subjected to germination conditions in order to germinate viable spores. Specifically, a quantity of the spore suspension was inoculated into brain-heart infusion medium to a concentration of 2.3×10⁵ spores/ml and incubated at 37° C. Aliquots of the germinating sample were removed after 1, 2, 3, and 4 hours and stained and analyzed essentially as described in Example 1. The results, shown as FL1-H vs. FL3-H plots, are shown in FIGS. 3B through 3E.

The effect of germination on the staining of the spores can be seen by comparing the results shown in FIGS. 3A through 3E. After one hour under germination conditions, a significant fraction of spores have germinated and, as a consequence, now exhibit increased fluorescence intensity after staining in both yellow (FL-1) and red (FL-2). By three hours, almost all the spores have germinated and the population of germinated spores is clearly distinguishable from the population of remaining ungerminated spores.

The fraction of spores that were viable, determined from the samples germinated for 3 hours, was calculated to be 96% of the total spores.

### EXAMPLE 5

### Comparison to Plate-Count Methods

The concentration of spores in the sample described in Example 4 was calculated from the flow cytometry data, as described in Example 1. The calculated spore concentration was 3.0×10⁶/ml. This determination of spore concentration, determined by flow cytometry, can be compared with the spore concentration determined by limiting dilution plate counts.

Determining spore concentration based on limiting dilution plate counts is well known in the art. As used herein, spore concentrations were determined as follows. Ten-fold serial dilutions of the sample were prepared (1:10, 1:100, 1:1000, 1:10000). From each of these dilutions, 100, 200, 300, or 400 μL aliquots were plated on Trypticase™ soy agar (BD Diagnostic Systems, Sparks, M.D., Catalog No. 1982324) and incubated for 1 to 2 days at 37° C. The incubation allows for both germination of the spores and subsequent growth of a detectable colony. Plates with well resolved colonies were counted and the initial concentration of viable spores was calculated from the count and the dilutions.

As noted above, the spore concentration of the initial spore suspension used in Example 4 was determined by plate count to be about 1.3×10⁶ viable Bacillus subtilis spores/ml. Thus, flow cytometric determination resulted in a higher estimate (3.0×10⁶/ml vs. 1.3×10⁶/ml) of spore concentration. Based on a number of experiments (data not shown), it appears that flow cytometric determination consistently results in a higher estimate, typically two- to three-fold higher, of spore concentration than obtained by plate-count methods. The cause of the difference in results obtained by the two methods is not known.

What is claimed is:

1. A method of detecting viable spores in a sample, said method comprising the steps of:
a) contacting an aliquot of said sample with a permeant dye and an impermeant dye to produce a stained ungerminated sample;
b) treating an second aliquot of said sample to germination conditions,
c) contacting said second aliquot with a permeant dye and an impermeant dye to produce a stained germinated sample;
d) flow cytometrically analyzing said stained ungerminated sample to obtain a ungerminated data set;
e) flow cytometrically analyzing said stained germinated sample to obtain a germinated data set; and
f) comparing said ungerminated and germinated data sets to detect viable spores in said sample.

2. The method of claim 1, wherein said spores are fungal.

3. The method of claim 1, wherein said spores are bacterial.

4. The method of claim 1, wherein said method further comprises contacting said first aliquot and said second aliquot with a calibration composition prior to or during said steps d) and e) such that said stained ungerminated sample and said stained germinated sample each include a calibration composition.

5. The method of claim 5, wherein said calibration composition is a microparticle composition.

6. The method of claim 4, wherein said ungerminated data set and said germinated data set are quantitative data sets.

7. The method of claim 6, wherein said comparing step f) comprises comparing said quantitative ungerminated data set and said quantitative germinated data set to detect either the number or concentration of viable spores in said sample.

8. The method of claim 1, wherein said contacting step (a) and said contacting step (c) further comprise contacting said sample with at least one surfactant.

9. The method of claim 1, wherein said contacting step (a) and said contacting step (c) further comprise contacting said sample with at least one chelating agent.

10. A method of determining the number or concentration of viable spores in a sample, said method comprising:
a) contacting an aliquot of said sample with:
i) a permeant dye;
ii) an impermeant dye;
iii) at least one surfactant; and
iv) a calibration microparticle composition;
to produce a stained ungerminated sample;
b) treating an second aliquot of said sample to germination conditions,
c) contacting said second aliquot with:
i) a permeant dye;
ii) an impermeant dye;
iii) at least one surfactant; and
iv) a calibration microparticle composition;
to produce a stained germinated sample;
d) flow cytometrically analyzing said stained ungerminated sample to obtain a ungerminated data set;
e) flow cytometrically analyzing said stained germinated sample to obtain a germinated data set; and
f) comparing said ungerminated and germinated data sets to determine the number or concentration of viable spores in said sample.

11. The method of claim 10, wherein said calibration microparticle composition is a plurality of fluorescent microbeads.

12. The method of claim 10, wherein said spores are fungal.

13. The method of claim 10, wherein said spores are bacterial.

14. A method of determining the effectiveness of a sterilization process, said method comprising:
subjecting a sample to said sterilization process;
determining the number or concentration of viable spores in said sample using the method of claim 10; and
comparing said determined number or concentration to a control to determine the effectiveness of said sterilization process.

15. A kit for detecting viable spores in a sample, said kit comprising:
a) a permeant dye;
b) an impermeant dye; and
c) a substrate comprising instructions for practicing the method of claim 1.

16. The kit of claim 15, wherein said kit further comprises a calibration composition.

17. The kit of claim 16, wherein said calibration composition is a microparticle composition.

18. The kit of claim 15, wherein said kit further comprises a surfactant.

19. A system for detecting viable spores in a sample, said system comprising:
a) a permeant dye;
b) an impermeant dye;
c) a flow cytometer;
d) computer-readable medium containing programming for practicing the method of claim 1; and
e) a substrate comprising instructions for practicing the method of claim 1.
